(19) United States
(12) Patent Application Publication
(10) Pub. No.: US 2007/0173842 A1
(43) Pub. Date: Jul. 26, 2007

(54) Device and Method for the Placement of Spinal Fixators

(76) Inventor: M. S. Abdou, San Diego, CA (US)

Correspondence Address: FISH & RICHARDSON, PC, P.O. BOX 1022, MINNEAPOLIS, MN, 55440-1022, US

(21) Appl. No.: 11/564,816

(22) Filed: Nov. 29, 2006

Related U.S. Application Data
(60) Provisional application No. 60/740,301, filed on Nov. 29, 2005.

(51) Int. Cl. A61F 2/30

(52) U.S. Cl. 606/69

(57) ABSTRACT

Devices and methods are adapted for the distraction of a disc space that is situated between a first vertebral body attached to a previously implanted bone fixator and a second vertebral bone that is not attached to the bone fixator. A distraction member is attached onto the previously implanted fixator while the fixator remains attached to the first vertebral body such that the fixator is not removed from the first vertebral body. A distraction force is then onto the fixator via the distraction member to distract the disc space

### REFERENCE TO PRIORITY DOCUMENT

This application claims priority of co-pending U.S. Provisional Patent Application Ser. No. 60/740,301, filed Nov. 29, 2005. Priority of the aforementioned filing date is hereby claimed and the disclosure of the Provisional Patent Application is hereby incorporated by reference in its entirety.

### BACKGROUND

The present disclosure is directed at skeletal plating systems, components thereof, and method of implant placement. These systems are used to adjust, align and maintain the spatial relationship(s) of adjacent bones or bony fragments after surgical reconstruction of skeletal segments.

In spinal surgery, it is accepted that fusion of a specific segment will increase the load on, and the rate of degeneration of, the spinal segments immediately adjacent to the fused level. As the number of spinal fusion operations has increased, so has the number of patients who harbor diseased adjacent segments and who will develop pain, neurologic deficit and disability. To treat this condition, surgeons remove the degenerating tissues and then extend the fusion onto the adjacent motion segment.

The fusion of spinal segments is usually supplemented by the application of a bone fixation device. These devices are attached to the underlying vertebrae using bone screws or similar fasteners and act to support the bone and share the load while the fusion matures. At the time of fusion extension, the fixation device is often found overlying the bony segment to be fused and limiting access to the underlying bone. In regions of small bone size, such as the cervical spine, the fixation device overlies the majority of the bone surface and prevents the surgeon from reaching the underlying bone.

In extending a fusion within the cervical spine, the original fixator (usually a plate and/or rod-based fixation device) must be removed in order to access the underlying bone. After device removal, distraction screws are placed into the vertebral bodies on each side of the diseased disc space. Using these screws, the disc space is distracted and opened, the degenerated disc is removed and a bone graft is placed into the evacuated disc space. A new bone fixation device is then applied across the newly fused disc space. Depending on the surgeon's preference, the new fixation device may extend across the newly fused vertebral levels alone or it may also incorporate one or more of the previously fused levels.

More recently, degeneration at the adjacent spinal segment has been treated with the removal of the diseased disc and its replacement with a mobile prosthesis, such as the artificial disc. While this approach avoids fusion, excision of the degenerated disc and the placement of the disc prosthesis still require that the diseased disc space be temporarily distraction. Since the existing fixator covers the anterior aspect of at least one of the vertebral bodies adjacent to the degenerated disc, the device would, again, limit access to the underlying bone and prevent distraction screw placement. As with fusion, the existing fixation device must be removed before the degenerating adjacent disc space can be implanted with an artificial disc.

Removal of the existing fixation device is not benign. Device removal necessitates re-exposure of the operative field from the initial fusion procedure and requires re-dissection through the scarred tissues at that site. Re-dissection increases the likelihood of tracheal, esophageal, pharyngeal, nerve and blood vessel injury and significantly increases the risk of post-operative swallowing difficulties, voice loss, stroke and other disabilities. It also increases the risk of bleeding and infection as well as increasing the length of the operation.

While plate removal is problematic in fusion extension procedures, the need to replace the existing plate with a longer one can be used to partly justify that risk. In procedures that implant an artificial disc, plate extension is not required. In these operations, placement of the distraction screw is the sole justification for plate removal.

### SUMMARY

A device and method that would permit distraction of the disc space adjacent to fused segments without fixation device removal would be clearly advantageous. It would completely eliminate the numerous risks of fixator removal and reduce the overall operative risk to the patient. Disclosed are multiple device embodiments and methods of use that accomplish this goal.

In the first embodiment, a distraction member is placed into a threaded aperture within the fixator. The distraction member is adapted to interact with a complimentary receptacle on a distraction platform and transmit the force produced by the platform onto the implanted bone fixator and the underlying bone to which the fixator is attached. In a second embodiment, a distraction member with a central receptacle is attached onto a threaded aperture within the fixator. The member is adapted to receive within its central receptacle a complimentary protrusion from the distraction platform. When coupled, the distraction member transmits the force produced by the distraction platform onto the implanted bone fixator. In another embodiment, the distraction member is adapted to be retained within a non-threaded aperture within the fixation device. While not separately illustrated, the distraction member assembly may be a male adapter that resides within a receptacle of the distraction platform or it may contain a central receptacle that accepts a complimentary protrusion of the distraction platform.

In another embodiment, a distraction member is attached onto one or more bone screws of the bone fixation device and serve as a coupling site for the distraction platform. Alternatively, one or more of the bone screws can be removed and the distraction member can be anchored directly to the underlying bone. Additional embodiments illustrate distraction members that can anchor onto larger openings within the fixator, such as the central opening, and serve to transmit the force produced by the distraction platform onto the implanted bone fixator. Alternatively, the distraction member may be advanced through the central opening of the fixator and attached directly onto the underlying bone so as to transmit the force of distraction directly onto the vertebra. In other embodiments, the distraction member is attached onto one or more walls or pillars of the fixator. While not separately illustrated for each embodiment, it is understood that the distraction member assembly may be a male adapter that resides within a receptacle of the distraction platform or it may contain a central receptacle that accepts a complimentary protrusion from the distraction platform.

In additional embodiments, a distraction platform is adapted to directly couple with the bone fixator or the underlying bone by preferably, but not necessarily, using one or more of the previously disclosed attachment methods. By permitting direct distractor platform to fixation device coupling, the need to place a separate distraction member before placement of the distraction platform is removed.

In one aspect, there is disclosed a method for the distraction of a disc space that is situated between a first vertebral body attached to a previously implanted bone fixator and a second vertebral bone that is not attached to the bone fixator. The method comprises attaching a distraction member onto the previously implanted fixator while the fixator remains attached to the first vertebral body such that the fixator is not removed from the first vertebral body; and exerting a distraction force onto the fixator via the distraction member to distract the disc space.

In another aspect, there is disclosed a method of distracting a disc space between a first vertebral body and a second vertebral body, comprising: attaching a distraction member to a fixator that is implanted onto the first vertebral body while the fixator remains implanted on the first vertebral body, wherein the second vertebral body is not attached to the fixator; and distracting the disc space by exerting a distraction force onto the distraction member.

In another aspect, there is disclosed a bone distraction device, comprising: a member having a first portion and a second portion, the first portion adapted to couple to a bone fixator attached to a bone such that the first portion remains coupled to the bone fixator during distraction of a disc space, the second portion adapted to coupled onto a distractor platform that produces a distraction force sufficiently strong to distract a disc space, wherein the distraction device is adapted to transmit the distraction force produced by the distraction platform onto the implanted bone fixator and the bone to which the fixator is attached while the first portion remains coupled to the bone fixator.

In another aspect, there is disclosed a bone distraction device, comprising: a member having a first portion and a second portion, the first portion adapted to couple to a bone fixator attached to a bone such that the first portion remains coupled to the bone fixator during distraction of a disc space, the second portion adapted to couple to a neighboring bone that is not attached to the fixator so that at least one disc space is situated between the bone attached to the fixator and the neighboring bone not attached to the fixator, wherein the distraction device is adapted to exert a force across the disc space while the first portion remains coupled to the bone fixator.

In another aspect, there is disclosed a method for the distraction of a disc space that is situated between a first vertebral body attached to a previously implanted bone fixator and a second vertebral bone that is not attached to the bone fixator, comprising: attaching a distraction member onto an aperture of the previously implanted fixator while the fixator remains attached to the first vertebral body such that the fixator is not removed from the first vertebral body; and exerting a distraction force onto the fixator via the distraction member to distract the disc space.

In another aspect, there is disclosed a method for the distraction of a disc space that is situated between a first vertebral body attached to a previously implanted bone fixator and a second vertebral bone that is not attached to the bone fixator, comprising: clamping a distraction member onto the previously implanted fixator while the fixator remains attached to the first vertebral body such that the fixator is not removed from the first vertebral body; and exerting a distraction force onto the fixator via the distraction member to distract the disc space.

Other features and advantages should be apparent from the following description of various embodiments, which illustrate, by way of example, the principles of the disclosed devices and methods.

### BRIEF DESCRIPTION OF THE DRAWINGS

FIG. 1 shows a plate attached to three vertebral bodies for fixating and fusing the three vertebral bodies.

FIG. 2 shows an exemplary embodiment of a distraction member that mates with one of the threaded apertures of the plate.

FIG. 3 shows the plate attached to three vertebral bodies and a bone screw removed from the plate.

FIG. 4 shows the distraction member readied for coupling to the open aperture of the plate.

FIG. 5 shows an exemplary distractor device.

FIG. 6 shows the distractor device after coupling to the distraction member and distraction screw.

FIG. 7 shows a female embodiment of the distraction member that includes an internal passageway that receives a portion of a distractor device.

FIG. 8A shows a distraction member that is adapted for attachment to a non-threaded borehole of a plate.

FIG. 8B shows an enlarged view of both the distal region and proximal region of the distraction member of FIG. 8A.

FIG. 9 shows the distraction member of FIGS. 8A/8B attached to the plate.

FIG. 10 shows another embodiment of a distraction member that is adapted to attach to a plate that has been attached to a vertebral body.

FIG. 11 shows the distraction member of FIG. 10 attached to the plate.

FIGS. 12 and 13 show another embodiment of the distraction member that is configured for attachment to a single bone screw of the plate.

FIGS. 14 through 16 show another distraction member that can be attached to plates that are used with bone screws.

FIGS. 17-19 shows an alternate embodiment of a distraction member.

FIG. 20 shows an enlarged view of an attachment region of the distraction member of FIGS. 17-19 attached to a hole in the plate.

FIG. 21 shows a distraction member that is adapted to clamp onto the plate.

FIG. 22 shows the distraction member of FIG. 21 with a clamp member clamped onto the outer walls of the plate.

FIG. 23 shows an exploded view of the distraction member of FIG. 21.

FIGS. 24 and 25 show cross-sectional views of two embodiments of a clamp member of a distraction member clamped onto the plate.

FIGS. 26-28 show another embodiment wherein a distraction member has a pliers-like configuration.

FIG. 29 shows an embodiment of a distractor device.

FIGS. 30 and 31 show an alternate embodiment of a distraction member.

FIGS. 32A and 32B show alternate embodiments of bone fixators.

### DETAILED DESCRIPTION

Disclosed are devices and methods that permit distraction of a disc space adjacent to fixated vertebral body without removing the fixator (such as a plate) from the vertebral body. As described in detail below, one or more distraction members are attached to at least a portion of the plate and/or a portion of the underlying vertebral body while the plate is attached to the vertebral body. The distraction member provides a means of applying a distraction force to the plate and/or the vertebral body sufficient to permit distraction of a disc space. Various embodiments of distraction members that attach to the plate and/or the underlying vertebral bodies are described herein.

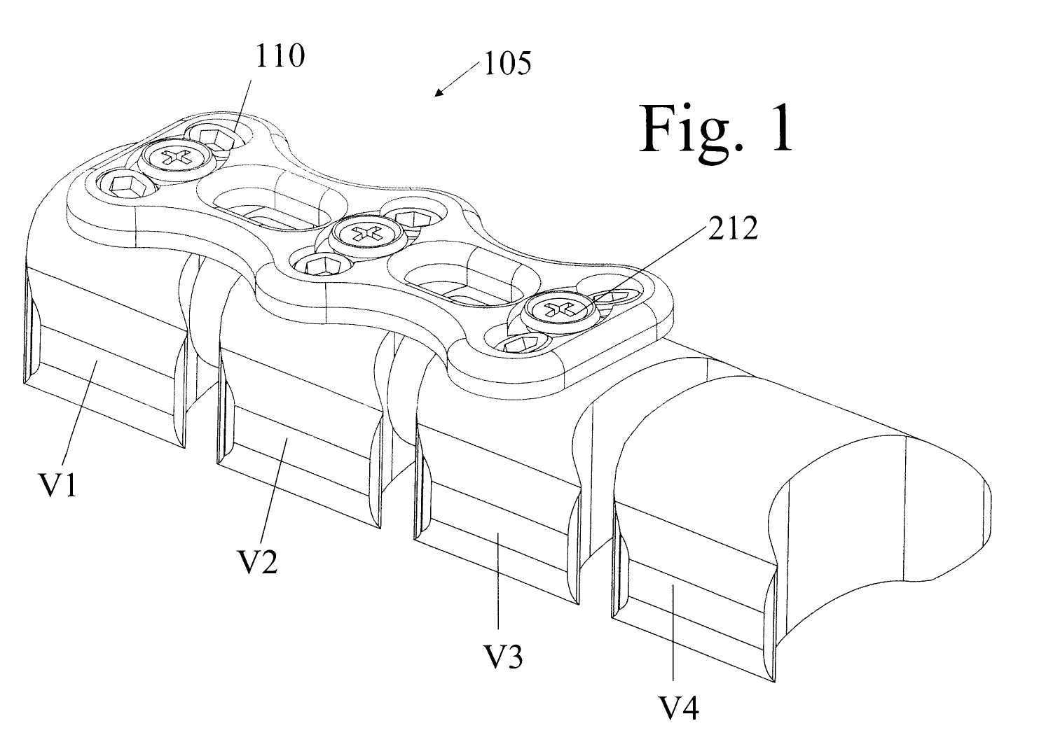

FIG. 1 shows a fixator comprised of a plate 105 attached to three vertebral bodies (V1, V2, and V3) for fixating and fusing the three vertebral bodies. The plate 105 includes several bone screws 110 that extend through bone screw apertures in the plate 105 to attach the plate to the underlying vertebral bodies. Bone grafts G are shown between the fused vertebras V1, V2, and V3. In the illustration, diseased disc A is shown adjacent to the fused bones and now requires surgical repair. The disc space A is between a fourth vertebral body V4 and one of the plated vertebral bodies wherein the vertebral body V4 is not attached to the plate.

Pursuant to the devices and methods described herein, a distraction member is attached to the plate and/or the underlying vertebral body for applying a distraction force. The distraction member can attach to various portions of the plate and/or the underlying bone. For example, the distraction member can attach to any threaded or non-threaded hole of the plate, the bone screw holes, the bone screws, the plate side walls or pillars, the central aperture of the plate, or any other plate aperture or channel.

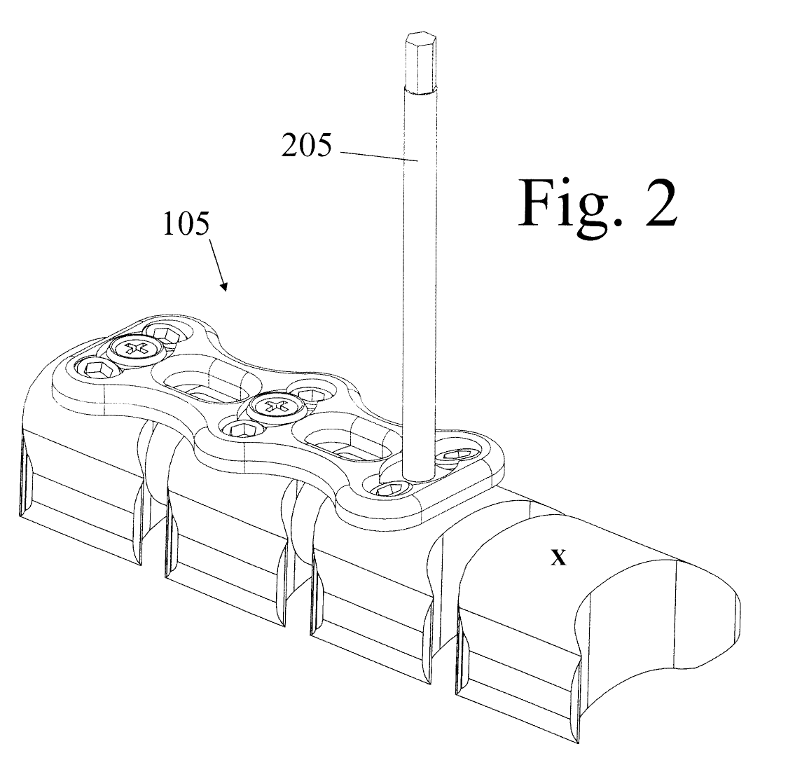

FIG. 2 shows an exemplary embodiment of a distraction member 205 that mates with one of the threaded apertures of the plate 105. In the embodiment of FIG. 2, the distraction member is an elongate structure having a threaded distal end that mates with a threaded aperture of the plate, as described in detail below. The distraction member can have other types of structures, as described further below.

The plate in FIGS. 1 and 2 is shown as being the type of plate described in U.S. Pat. Nos. 6,152,927 and 6,293,949, which are incorporated herein by reference. Such plates include threaded central apertures that are used to attach the plate's screw locking mechanism. It should be appreciated that the distraction members described herein can be used with other types of plates or fixators that are adapted to permit fixation of the spinal vertebras. Spinal plates typically include one or more apertures through which bone screws or other fasteners can be attached onto the underlying bone and some have additional apertures for other purposes. In some embodiments, the distraction members are adapted to couple to such apertures whether the apertures are threaded or non-threaded.

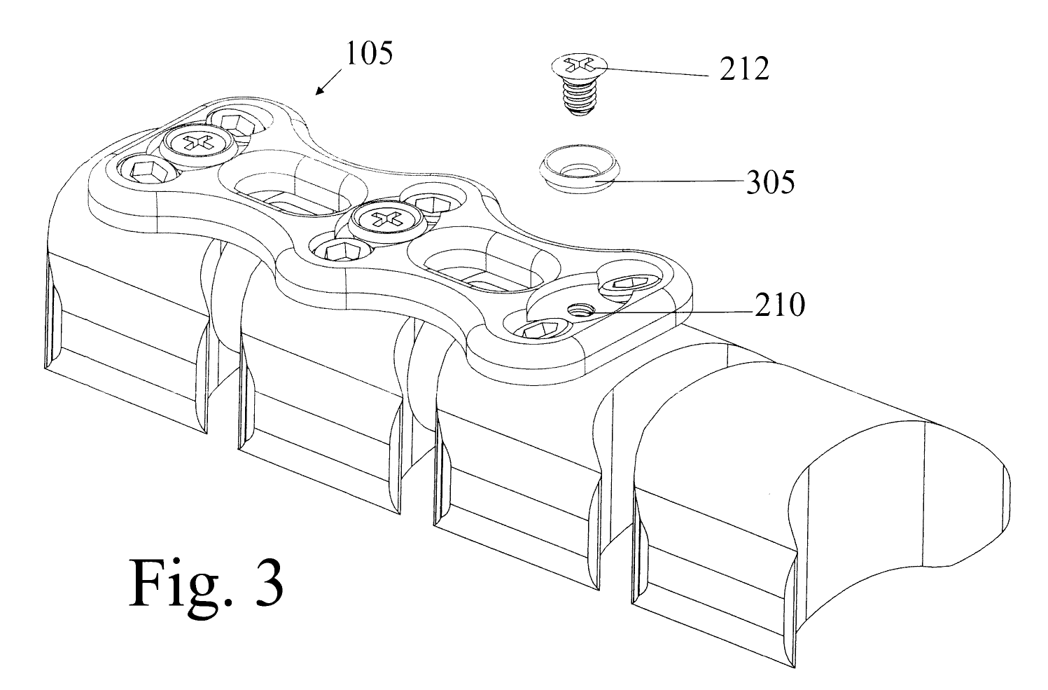

As mentioned, the distraction member 205 shown in FIG. 2 is an embodiment that is adapted to couple to a threaded aperture 210 in the plate. As shown in FIG. 3, prior to coupling the distraction member 205 to the plate, a screw 212 and corresponding washer 305 are removed from the plate 105 while the plate is attached to the vertebral bodies. Removal of the screw 212 exposes the threaded aperture 210. It should be appreciated that any other of the threaded apertures of the plate could be used. Moreover, as discussed, the distraction members described herein are not limited to use with threaded apertures as other embodiments are adapted for use with unthreaded apertures.

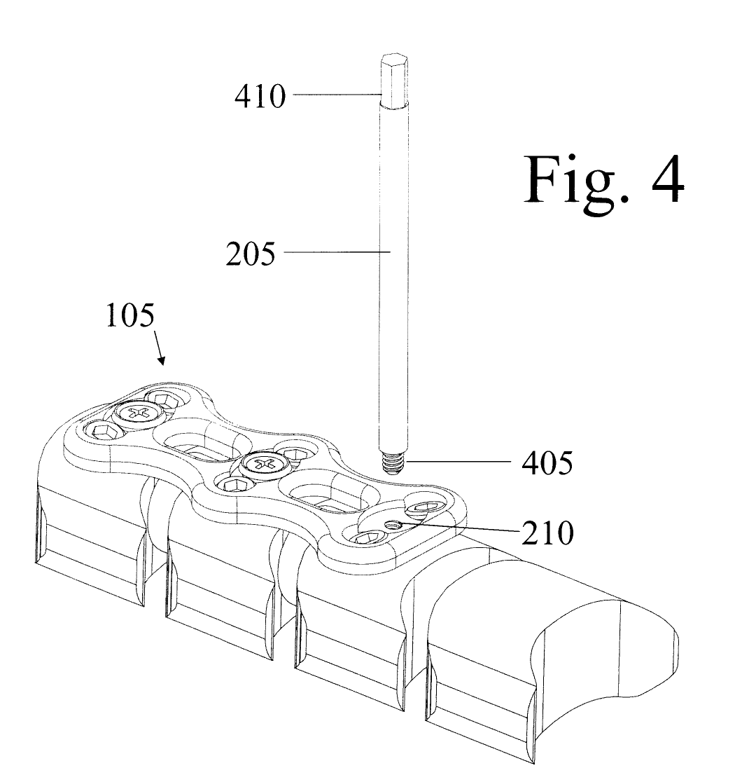

With reference now to FIG. 4, after removal of the screw 212, the distraction member 205 is next coupled to the plate 105. FIG. 4 shows the distraction member readied for coupling to the open aperture 210 of the plate 105. As mentioned, the distraction member 205 has a threaded distal region 405, which is sized and shaped for threading into the open aperture 210. The distraction member 205 can also include a coupling region 410 that is adapted to be coupled to a drive member for applying a drive force to the distraction member 205. The threaded distal region has threads that compliment the threads of the aperture 210. Thus, the distraction member 205 can be rotated into the aperture 210 for fixedly coupling the distraction member 205 to the plate, as shown in FIG. 2. Once the distraction member 205 has been attached to the plate, a conventional distraction screw can be placed onto an adjacent vertebral body, such as at location “X” in FIG. 2.

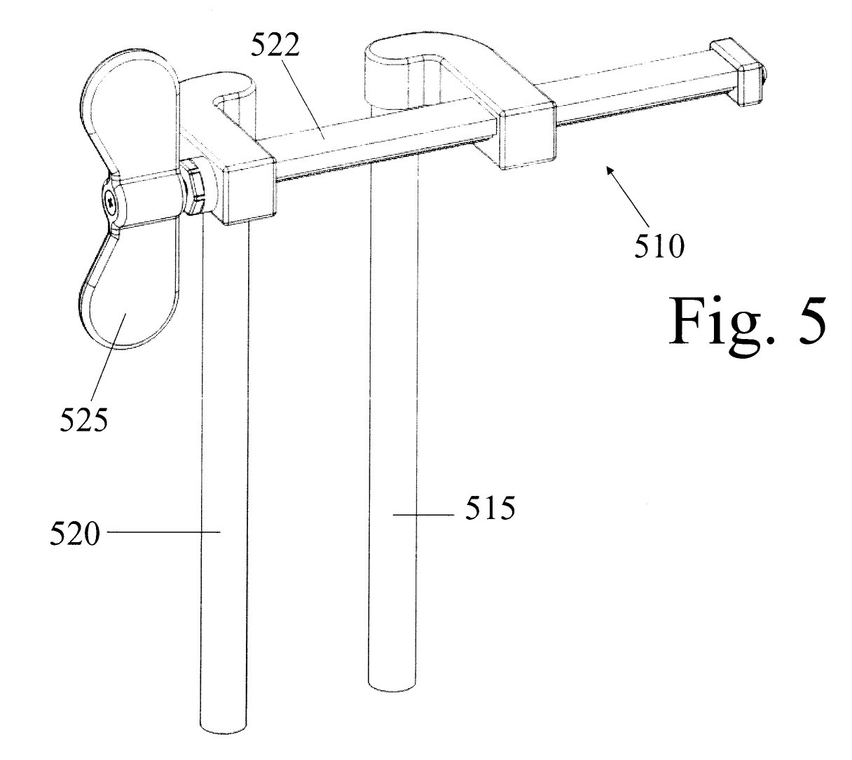

A distractor device is then used to engage the distraction member 205 and the conventional distraction screws placed at location “X” in FIG. 2. FIG. 5 shows an exemplary distractor device 510. The distractor device 510 includes a platform and one or more coupler components, such as a pair of elongate coupler arms 515 and 520 that are movably coupled each other via a linkage 522. An actuator 525 is actuated to cause the arms 520 and 515 to move toward or away from one another. The arms 520 and 515 are positioned such that they can be mated with the distraction member 205 and the convention distraction screw. In this regard, the arms 520 and 515 can have internal passageways that are sized and shaped to receive the distraction member 205 and distraction screw.

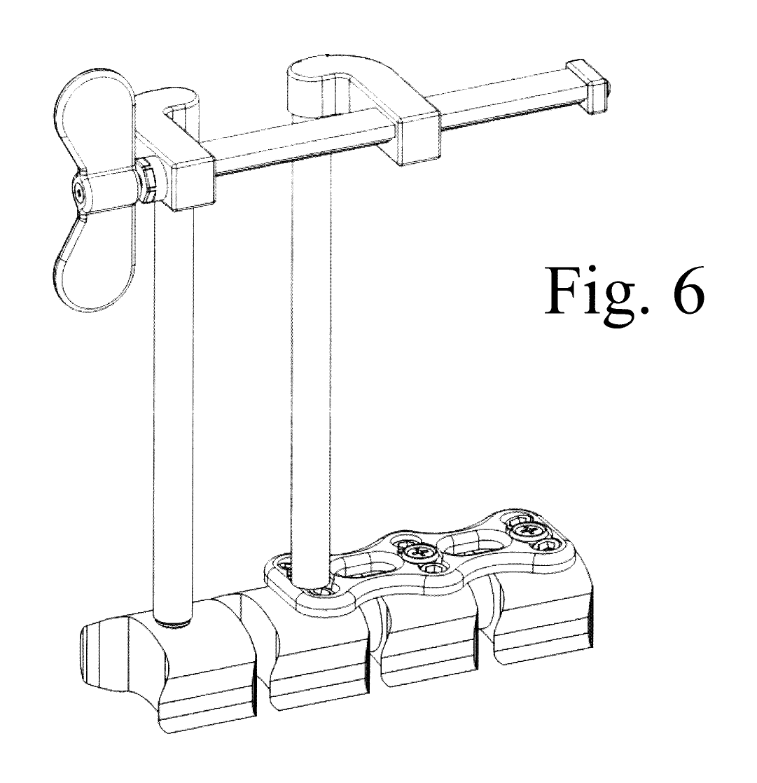

FIG. 6 shows the distractor device 510 after coupling to the distraction member 205 and distraction screw. The arm 515 has been placed over the distraction member 205 such that the coupling region 410 protrudes outwardly form the proximal end of the arm 515. With the distractor device 510 coupled as such, the distractor device is actuated to apply a distraction force (via the distraction member 205 and the conventional distraction screw) to the vertebral bodies and distract the diseased disc space. The disc space can then be evacuated and an orthopedic implant positioned in the evacuated space. In another embodiment, the vertebral body V4 is fused with one of the vertebral bodies V1, V2 and/or V3.

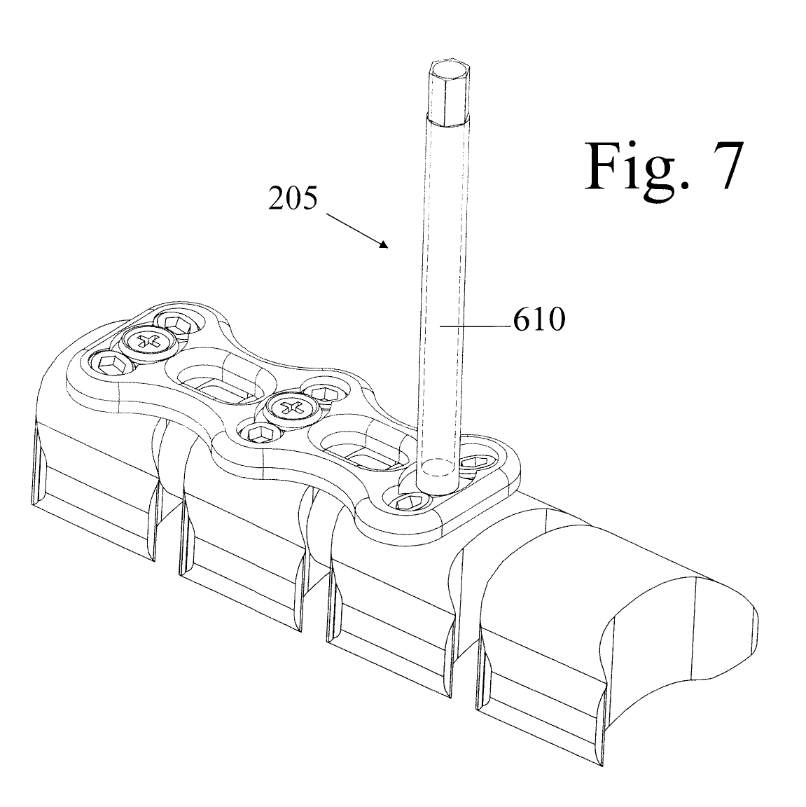

In the previously described embodiment, the distraction member 205 slides into the arm 515 of the distractor device in a male-female relationship. In another embodiment, the distraction member 205 has an internal passageway that receives the arm 515 of the distraction member. Thus, in the other embodiment, the distraction member 205 and arm 515 mate in a female-male relationship. FIG. 7 shows such an embodiment of the distraction member 205 that includes an internal passageway 610 that received a portion of a distractor device.

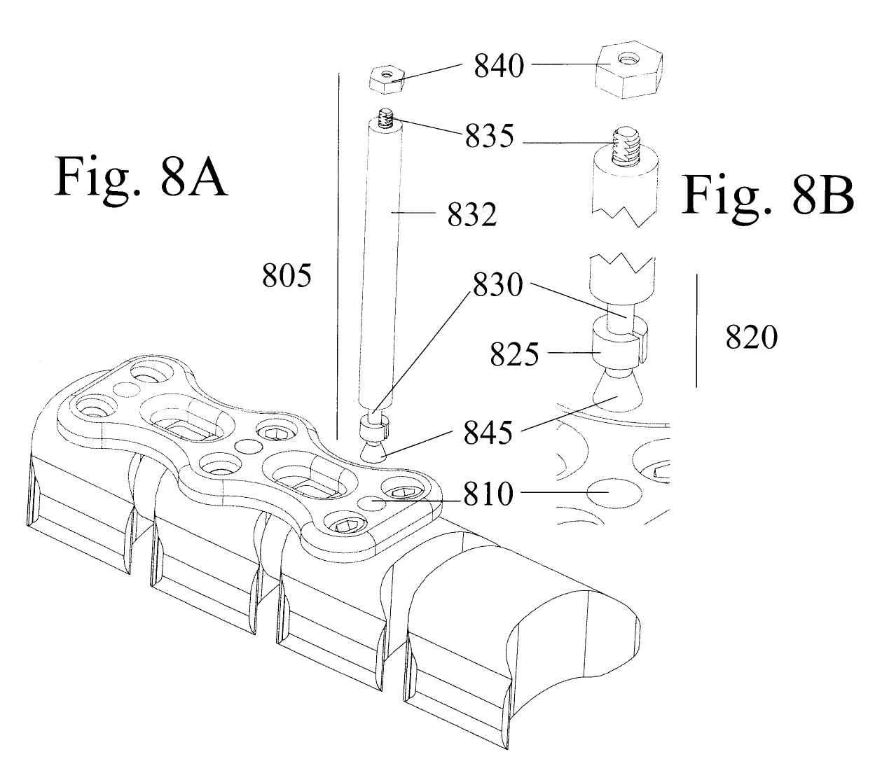

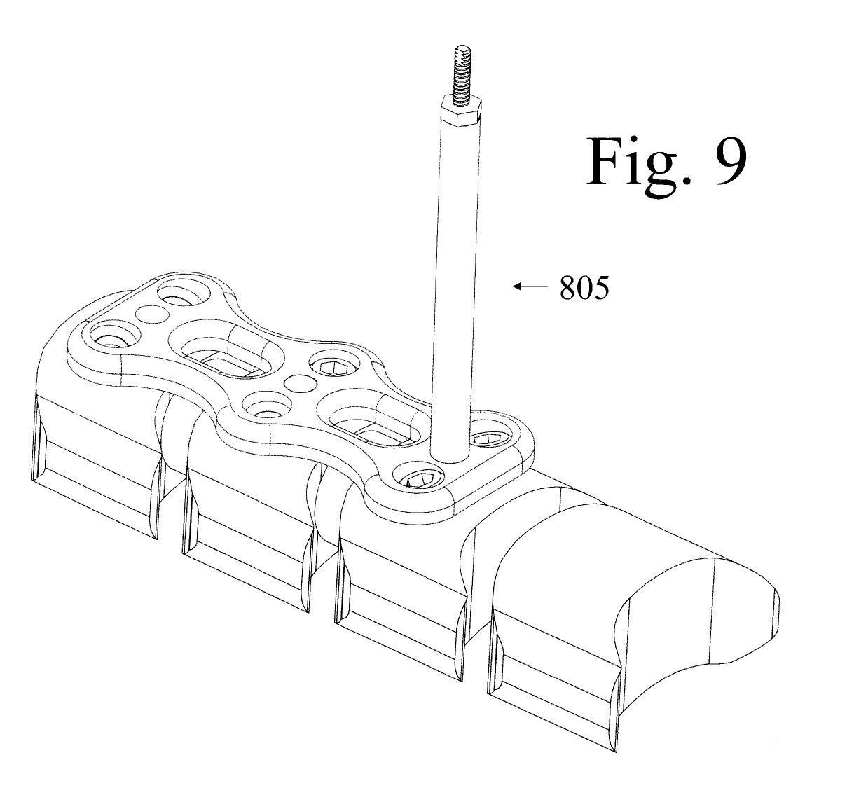

As mentioned, a distraction member can be adapted to be attached to a non-threaded aperture in a plate. FIG. 8A shows a distraction member 805 that is adapted for attachment to a non-threaded borehole 810 of a plate. FIG. 8B shows an enlarged view of both the distal region and proximal region of the distraction member 805. The distraction member 805 includes a plate attachment region 820 that is configured for attachment to the non-threaded aperture 810. In one embodiment, the plate attachment region 820 can be enlarged in size once positioned within the aperture 810 to thereby create a frictional engagement between the plate attachment region 820 and the aperture 810. The engagement force is sufficiently strong such that the distraction member 805 and the plate remain attached during distraction of the underlying vertebral body.

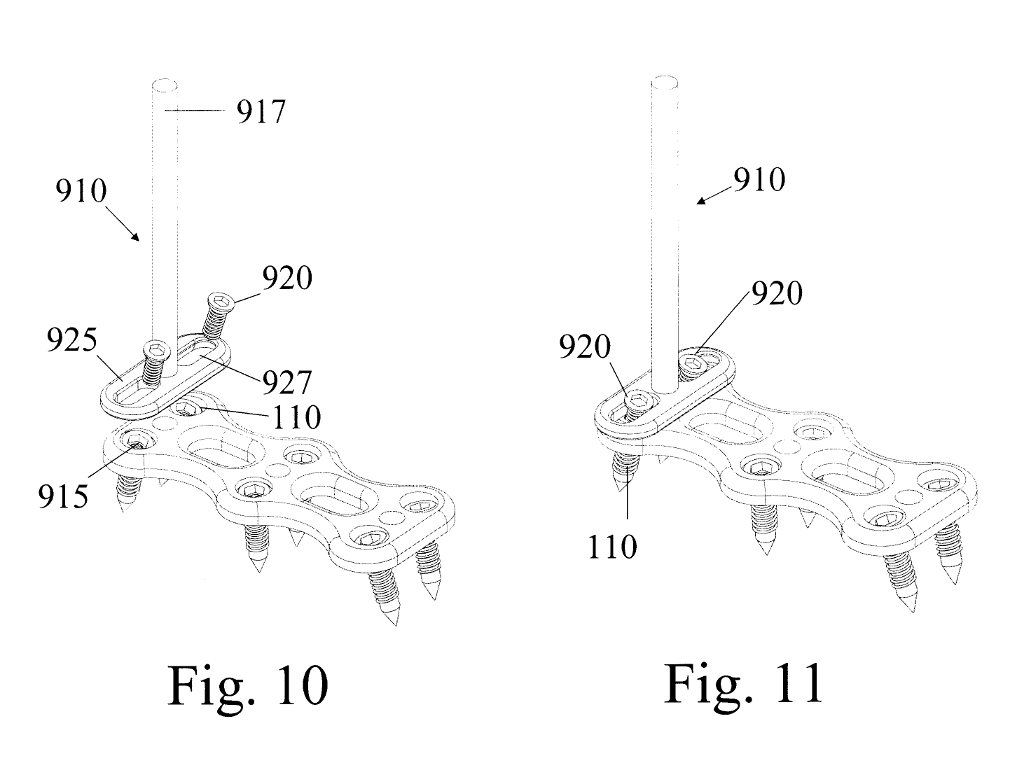

The enlargement of the plate attachment region 820 can be performed using various mechanisms. In the embodiment of FIGS. 8A and 8B, the plate attachment region 820 includes a collet 825 that is positioned over an internal member 830. The internal member 830 has a threaded proximal end 835 that mates with a tightening nut 840. A sloped or conical member 845 is located on the distal end of the internal member 830. In use, the plate attachment region 820 is positioned inside the non-threaded aperture 810. The nut 840 is then tightened, which pulls internal member 830 inward relative an outer member 832. This causes the conical member 845 to move into the collet 825 and expand the collet 825 outward. The collet 825 is expanded sufficiently large to create a frictional engagement between the collet 825 and the inside of the aperture 810 to secure the distraction member 805 to the plate. FIG. 9 shows the distraction member of FIGS. 8A/8B attached to the plate. As before, a distractor device is then used to engage the distraction member and a distraction screw (in either a male-female or female-male relationship) and a distraction force is applied.

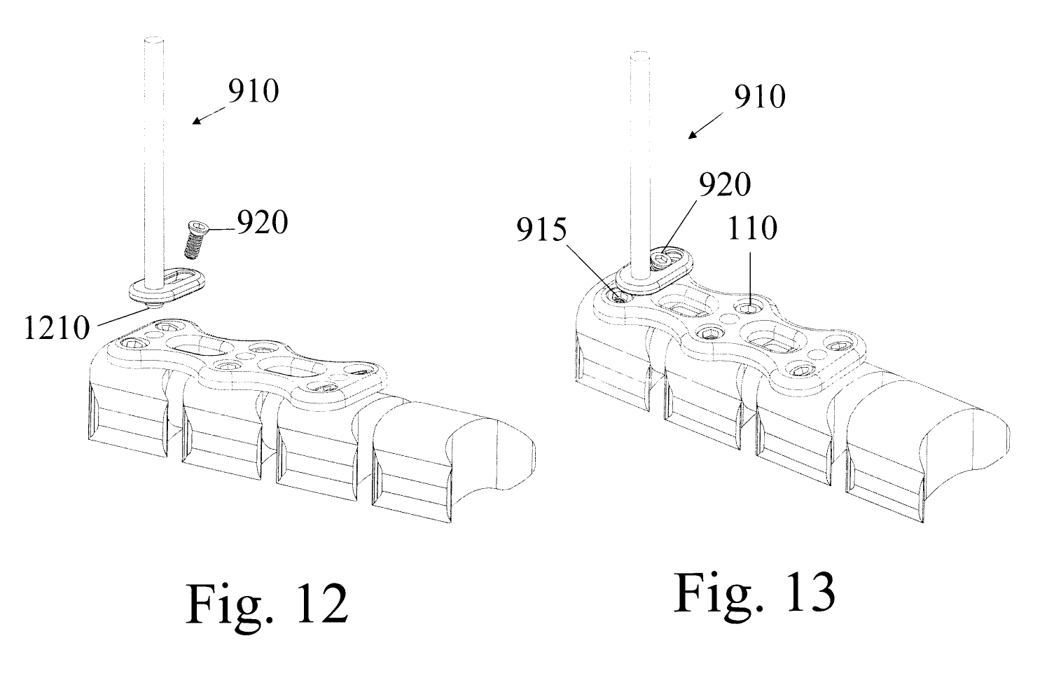

FIG. 10 shows another embodiment of a distraction member that is adapted to attach to a plate that has been attached to a vertebral body. In this embodiment, the distraction member 910 is configured to attach to a bone screw 110 that is positioned in the plate. The distraction member 910 includes an elongate portion 917 and an attachment region 925 that has a pair of openings 927 that are sized to receive threaded shafts of attachment screws 920. The attachment region 925 and the openings 927 are sized to be positioned over one or more bone screws 110 of the plate. The heads of the bone screws 110 have threaded bores 915 that are sized to receive the complimentary-threaded shafts of the attachment screws 920 of the distraction member 910.

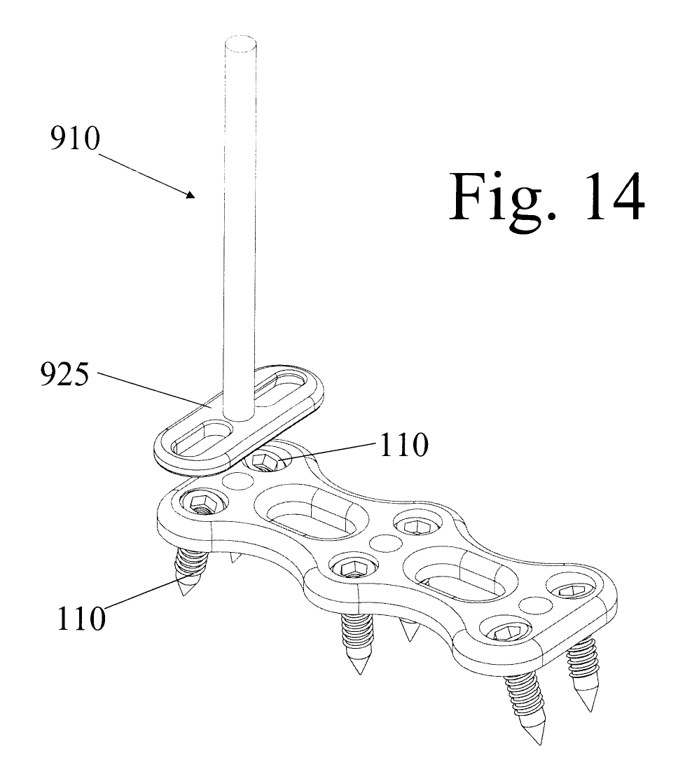

In use, the attachment region 925 of the distraction member 910 is positioned over the bone screws 110 of the plate. The attachment screws 920 are then threaded into the threaded bores 915 of the heads of the bone screws 110. In this manner, the distraction member 910 is secured to the plate via the bone screws 110. FIG. 11 shows the distraction member 910 attached to the plate. For clarity of illustration, the underlying vertebral body is not shown in FIG. 11. As discussed above, a distractor device is then used to engage the distraction member 910 and a distraction screw (in either a male-female or female-male relationship) and a distraction force is applied.

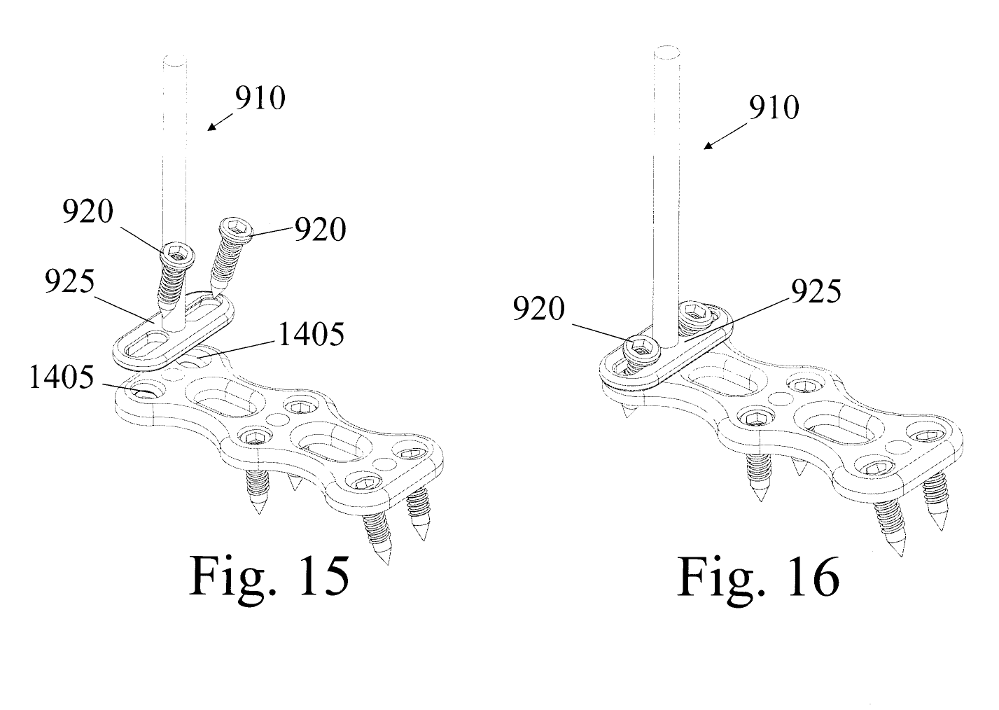

In the embodiment of FIGS. 10 and 11, the distraction member 910 attaches to two bone screws 110 of the plate. It should be appreciated that the distraction member 910 could be configured for attachment to only a single bone screw or to more than two bone screws. For example, FIGS. 12 and 13 show another embodiment of the distraction member 910 that is configured for attachment to a single bone screw 110 of the plate. As in the embodiment of FIGS. 10 and 11, the distraction member 910 includes an attachment screw 920 that screws into a threaded bore 915 in the head of one of the bone screws 110 of the plate. With reference to FIG. 12, the attachment region 925 can include a bottom protrusion 1210 that fits into one of the apertures on the plate for properly positioning the distraction member 910 relative to the plate.

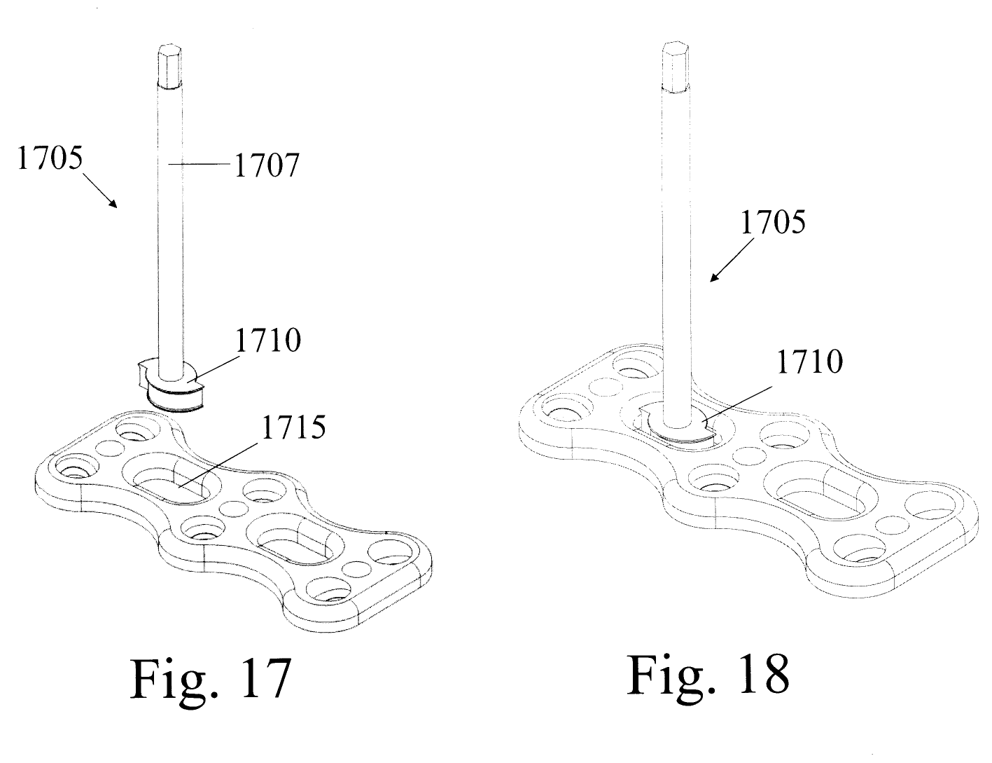

FIGS. 14 through 16 show a distraction member 910 that can be attached to plates having bone screws 110 that lack a threaded bore or engagement cavity within the head. As shown in FIG. 15, the bone screws 110 are removed from the plate and the underlying bone such that the apertures 1405 are exposed and unoccupied. As shown in FIG. 16, one or more attachment screws 920 are then inserted through the attachment region 925 and through the unoccupied apertures 1405 to secure the distraction member 910 to the underlying bone. (The underlying bone is not shown in FIGS. 14-16 for clarity of illustration.) As in the previous embodiments, once the distraction member 910 is attached to the plate or the bone, a conventional distractor screw is attached to the adjacent vertebral bodies. A distractor device is then used to apply a distraction force (via the distraction member 910 and the conventional distraction screw) to the vertebral bodies and distract the diseased disc space.

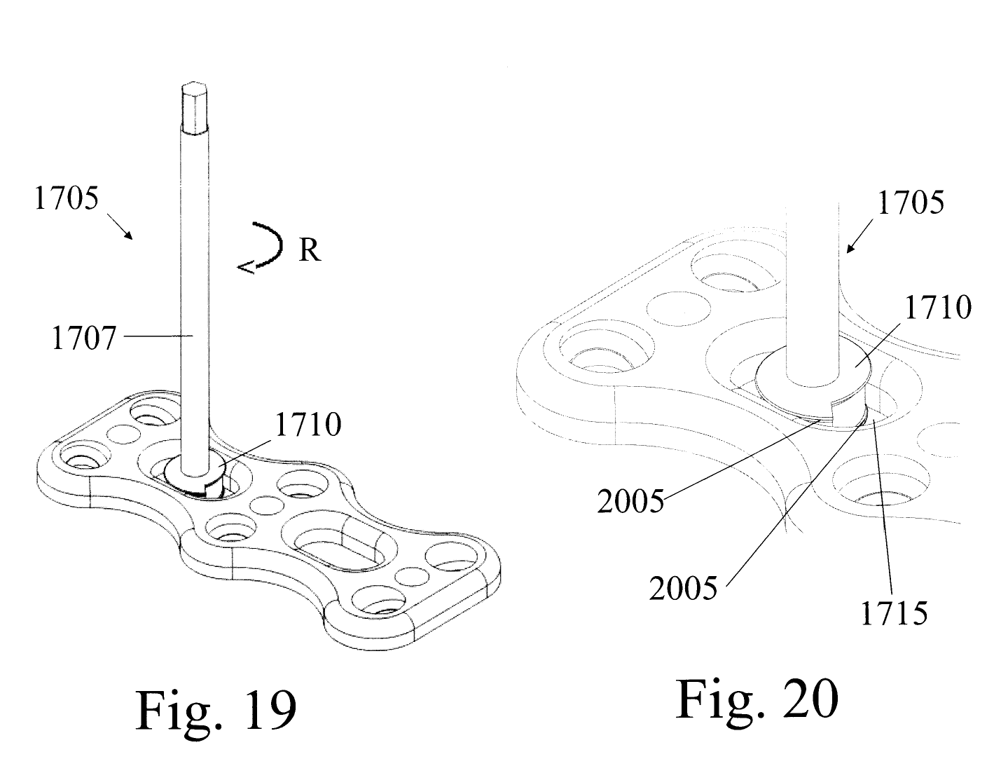

FIGS. 17-19 shows an alternate embodiment of a distraction member 1705. In this embodiment, the distraction member 1705 includes an elongate portion 1707 and a distal attachment region 1710 that is configured to attach to any opening in the plate, such as a central opening 1715 in the plate. The attachment region 1710 has an irregular or eccentric shape that fits within the opening 1715 when in a first orientation. The attachment region 1710 can be moved to a second orientation that causes engagement with the plate. For example, the attachment region 1710 can be rotated about an axis defined by the elongate portion such that a portion of the attachment region engages the side walls or other portion of the opening 1715. This forms an engagement that secures the distraction member 1705 to the plate.

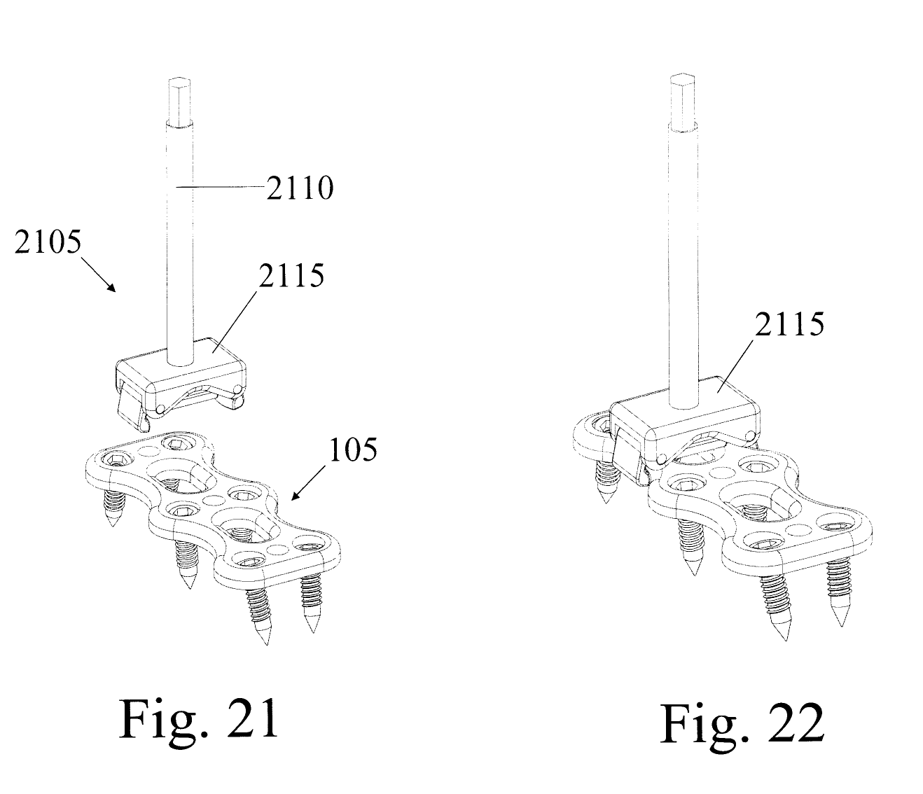

This is described in more detail with reference to FIGS. 18-20. As shown in FIG. 18, the attachment region 1710 is first positioned in an orientation such that it can be inserted into the aperture 1715. Next, as shown in FIG. 19, the attachment region 1710 is re-oriented, such as by rotating the attachment region as represented by arrow R in FIG. 19. The rotation causes the irregular shaped portions of the attachment region 1710 to engage at least a portion of the opening 1715. FIG. 20 shows an enlarged view of the attachment region 1710 of the distraction member 1705 attached to the hole 1715 in the plate. The attachment region 1710 includes upper and lower ledges 2005 that engage the upper and lower edges of the opening 1715 to thereby secure the distraction member 1705 to the plate. As in the previous embodiments, once the distraction member 1705 is attached to the plate, a conventional distractor screw is attached to the adjacent vertebral bodies. A distractor device is then used to apply a distraction force (via the distraction member 1705 and the conventional distraction screw) to the vertebral bodies and distract the diseased disc space.

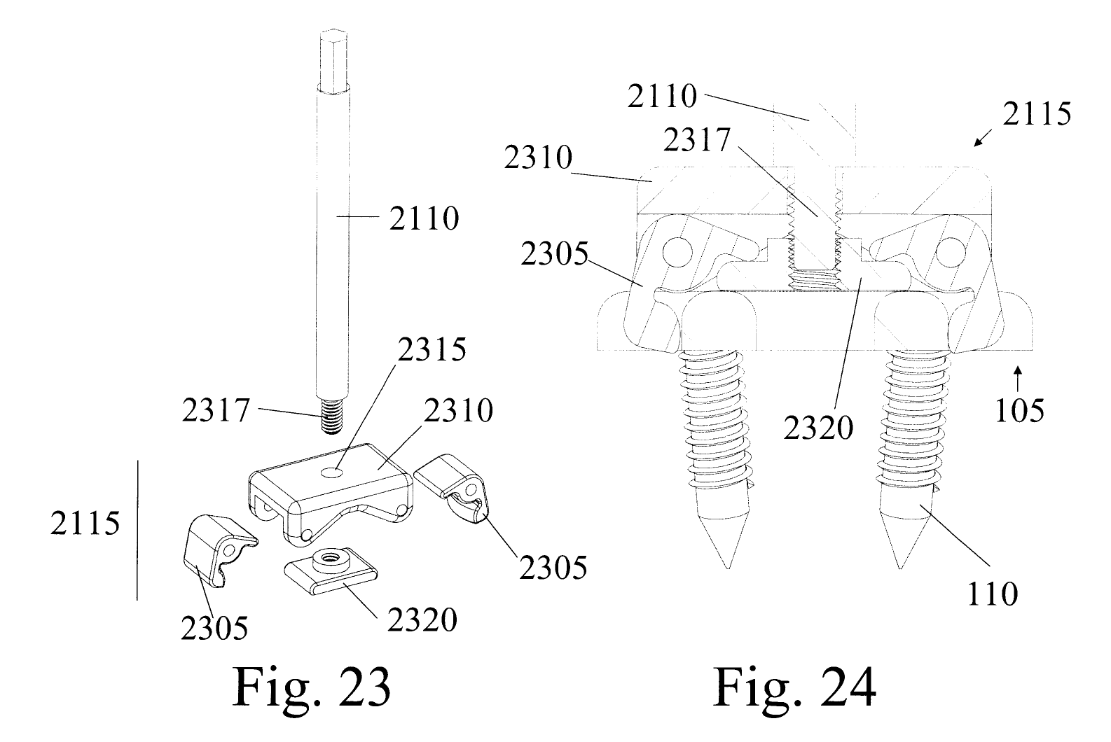

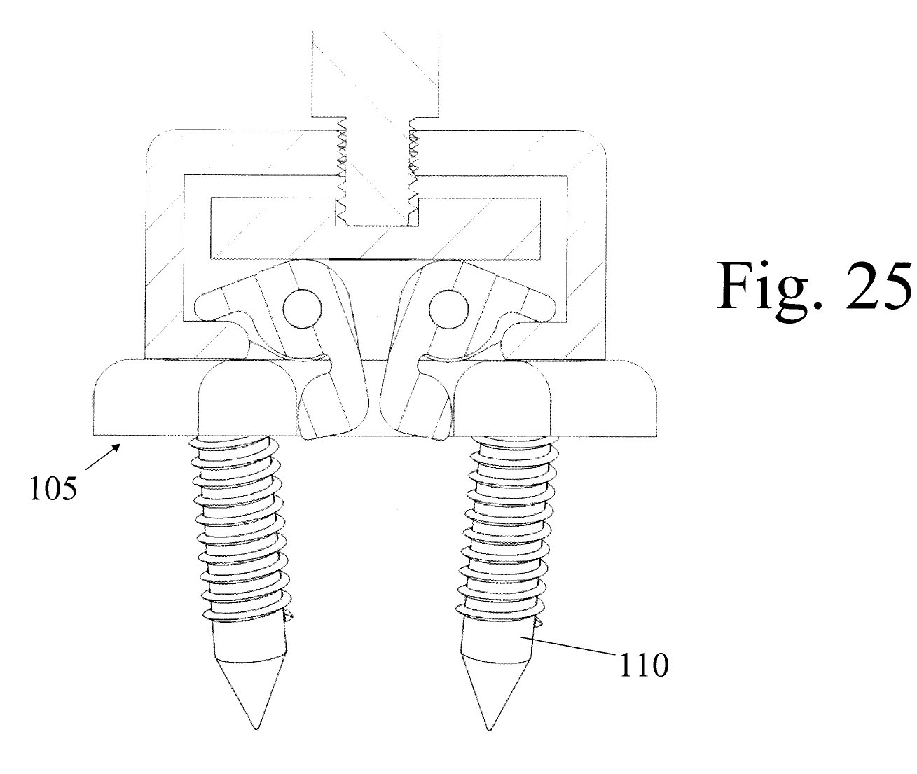

The distraction members described herein can be attached to various regions of the plate and/or the underlying vertebral bodies. For example, the distraction members can be configured to attach to the outer walls of the plate. FIG. 21 shows a distraction member 2105 that is adapted to clamp onto the outer walls of the plate 105. The distraction member 2105 includes an elongate member 2110 and a distal clamp member 2115 that clamps onto the plate 105. FIG. 22 shows the distraction member 2105 with the clamp member 2115 clamped onto the outer walls of the plate 105.

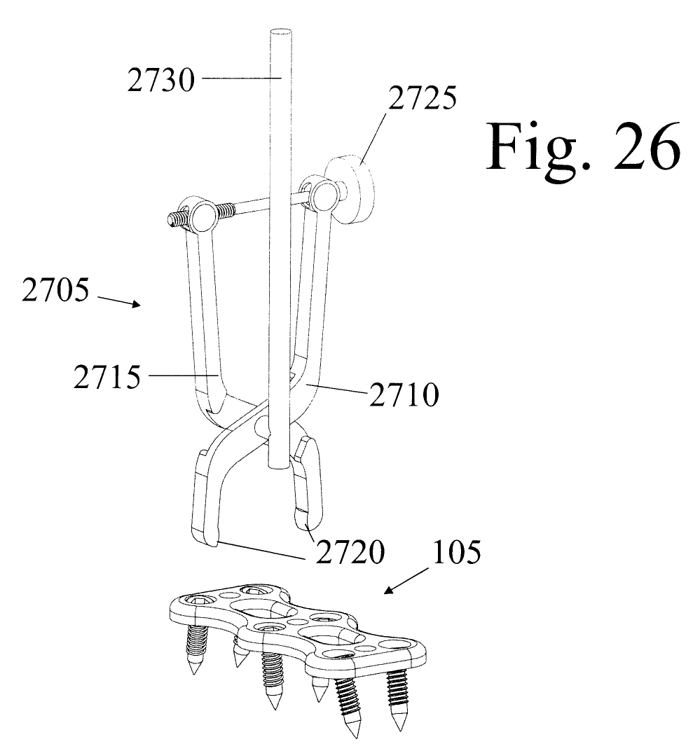

FIG. 23 shows an exploded view of the distraction member 2105. The clamp member 2115 includes a pair of clamp arms 2305 that are rotatably attached to a base 2310 (central attachment pins are not shown). The base has an aperture 2315 that receives the distal end 2317 of the elongate portion 2110. A lock component 2320 mates with the distal end 2317 of the elongate portion.

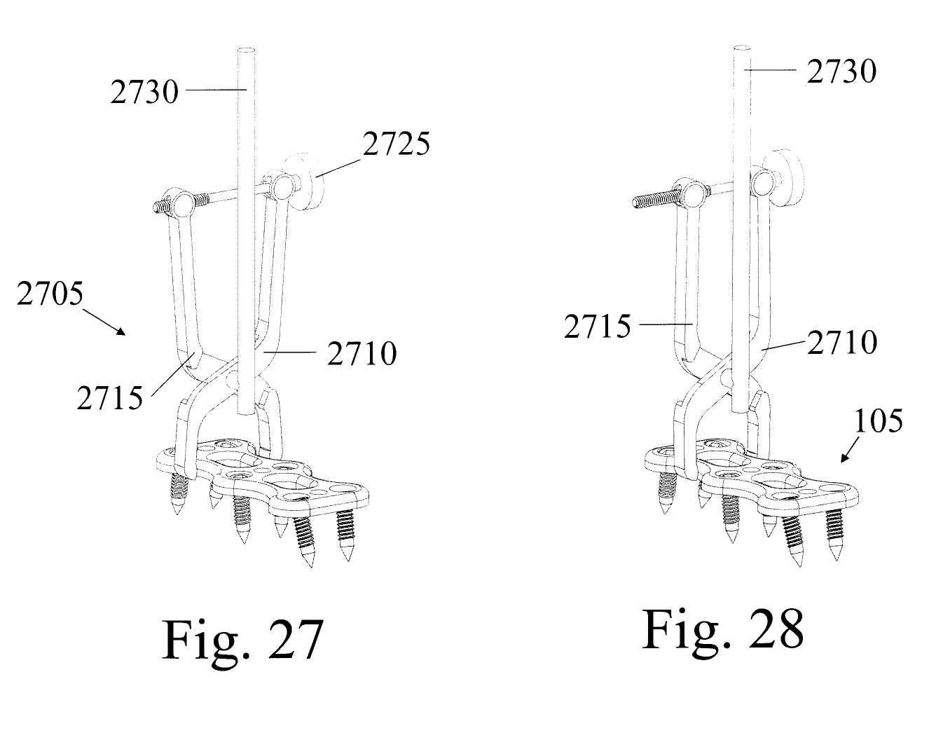

FIGS. 24 shows a cross-sectional view of the clamp member 2115 clamped onto the plate 105. Prior to clamping the clamp member onto the plate, the clamp arms 2305 are positioned adjacent the outer side walls of the plate 105. The lock component 2320 has an upper surface that engages a portion of the clamp arms 2305 to maintain the clamp arms in an open or closed state. With reference to FIG. 24, the elongate portion 2110 is rotated to cause the lock component 2320 to rise toward the base 2310 via the threaded engagement between the lock component 2320 and the threaded distal end 2317 of the elongate portion 2110. As the lock component 2320 rises, it pushes against a portion of the clamp arms 2305 to cause the clamp arms 2305 to close toward each other and clamp onto the outer walls of the plate 105. In this manner, the distraction member 2105 attaches to the plate 105.

The distraction member 2110 is capable of rigidly clamping onto the plate. As shown in FIG. 24, it may be used to attach onto the outer plate walls. Alternately, it may be sized to attach onto one plate column. In addition, the device may be configured to extend the clamp members outwardly and thus attach onto the inner walls of any aperture within the plate (such as the central plate aperture), as shown in FIG. 25. The device may alternately employ a female adapter to engage the distractor device, as previously discussed with respect to the other embodiments.

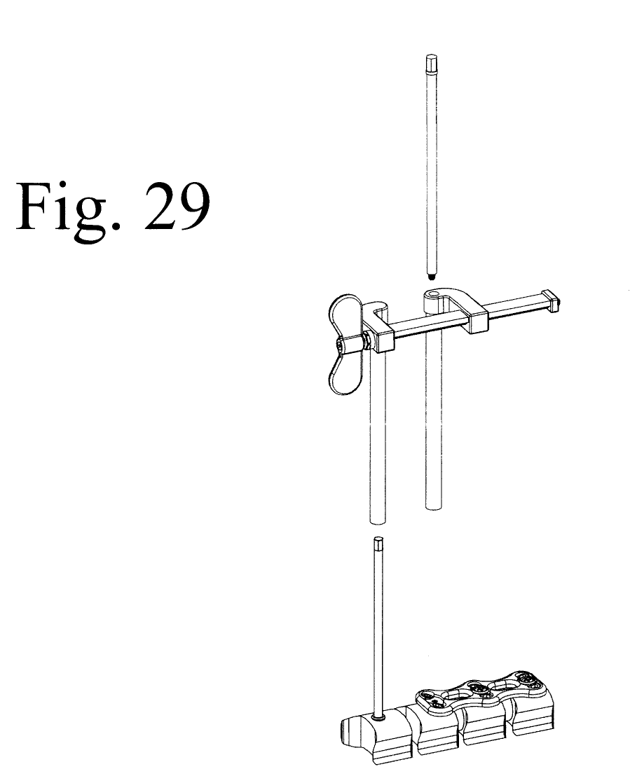

FIGS. 26-28 show another embodiment wherein a distraction member 2705 has a pliers configuration. The distraction member 2705 includes a first arm 2710 and a second arm 2715 that are rotatably attached to one another. The first and second arms include clamping regions 2720 that can be moved toward one another by manipulating an actuator 2725. The actuator 2725 is attached to a threaded rod that links the two arms to one another. In use, the clamping regions are positioned outside of the outer side walls of the plate, as shown in FIG. 27. The actuator 2725 is then actuated to move the clamping regions toward one another such that they clamp or otherwise grasp the plate therebetween, as shown in FIG. 28. The distraction member 2705 includes a solid elongate portion 2730 to which a distraction force can be applied once the distraction member 2705 has been attached to the plate.

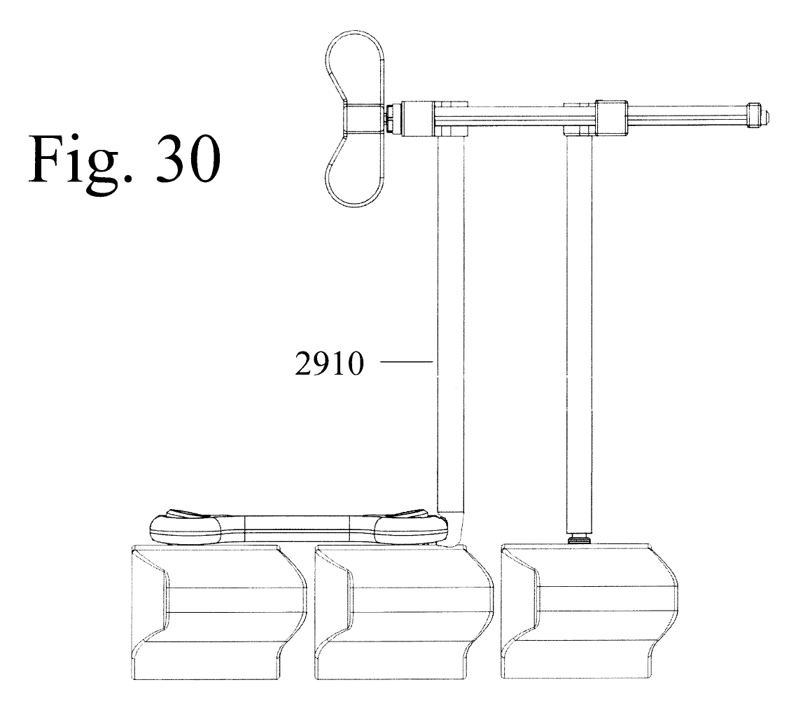

The preceding embodiments have illustrated how the distraction members attached onto an existing bone fixation plate using any threaded or non-threaded hole, the bone screw holes, the bone screws, the plate side walls or pillars, the central aperture or any other plate aperture or channel. As an alternative, a distractor device (such as the device shown in FIG. 5) can be modified such that any one of the distraction members described herein is integrally incorporated onto the device. Thus, the distractor device can be modified such that it can directly couple onto the plate. FIG. 29 show one such embodiment. Using this design, there is no need to place a distraction member on the plate distractor device placement. While the illustration shows the distractor device adapted to mate with a threaded plate bore, other features may be alternatively added to the distractor device so that it can engage any non-threaded plate hole, the bone screw holes, the bone screws, the plate side walls or pillars, the central aperture or any other plate aperture or channel.

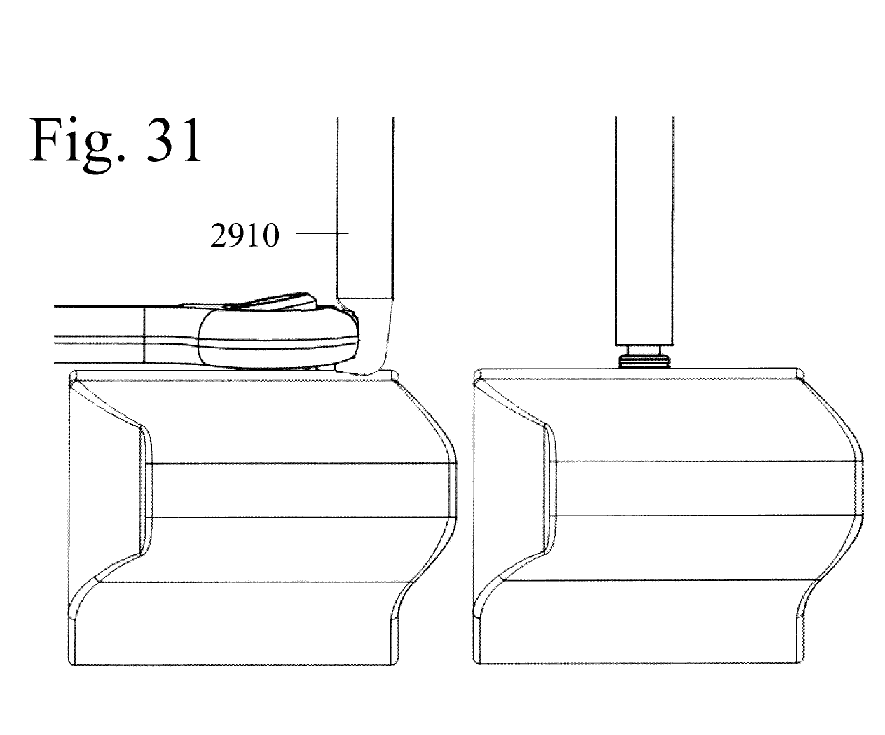

In another embodiment, shown in FIGS. 30 and 31, the distraction member has an arm 2910 that is configured to couple to an edge of the bone plate. As shown in FIG. 31, a distal region of the arm 2910 abuts an edge of the plate in a manner that permits the arm to exert a distraction force onto the plate while the arm remains attached to the plate.

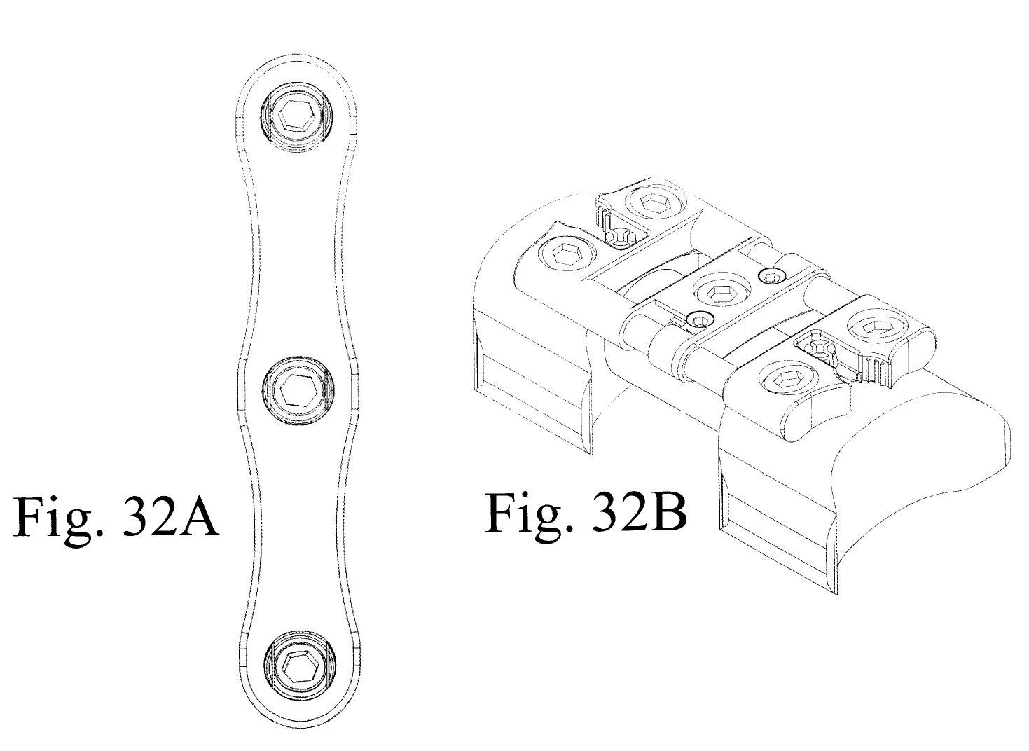

As mentioned, the fixator device is not limited to the type of plate shown in FIG. 1. FIGS. 32A and 32B show exemplary embodiments of other type of fixators that can be coupled to the distraction members described herein.

The disclosed devices or any of their components can be made of any biologically adaptable or compatible materials. Materials considered acceptable for biological implantation are well known and include, but are not limited to, stainless steel, titanium, tantalum, combination metallic alloys, various plastics, resins, ceramics, biologically absorbable materials and the like. Any components may be also coated/made with osteo-conductive (such as deminerized bone matrix, hydroxyapatite, and the like) and/or osteo-inductive (such as Transforming Growth Factor “TGF-B,” Platelet-Derived Growth Factor “PDGF,” Bone-Morphogenic Protein “BMP,” and the like) bio-active materials that promote bone formation. Further, a surface of any of the implants may be made with a porous ingrowth surface (such as titanium wire mesh, plasma-sprayed titanium, tantalum, porous CoCr, and the like), provided with a bioactive coating, made using tantalum, and/or helical rosette carbon nanotubes (or other carbon nanotube-based coating) in order to promote bone in-growth or establish a mineralized connection between the bone and the implant, and reduce the likelihood of implant loosening. In addition, any assembly or its components can also be entirely or partially made of a shape memory material or other deformable material.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

What is claimed is:

1. A method for the distraction of a disc space that is situated between a first vertebral body attached to a previously implanted bone fixator and a second vertebral bone that is not attached to the bone fixator, comprising:
attaching a distraction member onto the previously implanted fixator while the fixator remains attached to the first vertebral body such that the fixator is not removed from the first vertebral body;
exerting a distraction force onto the fixator via the distraction member to distract the disc space.

2. A method as in claim 1, further comprising:
evacuating the disc space; and
placing an orthopedic device in the evacuated disc space.

3. A method as in claim 1, further comprising:
evacuating the disc space; and
fusing the disc space.

4. A method as in claim 1, wherein the distraction member is attached to a threaded aperture of the fixator.

5. A method as in claim 1, wherein the distraction member is attached to a non-threaded aperture of the fixator.

6. A method as in claim 1, wherein the distraction member clamps onto the fixator.

7. A method as in claim 1, wherein the distraction member comprises an elongate member and further comprising mating the elongate member with a distractor device that exerts the distraction force.

8. A method as in claim 1, wherein the distraction member attaches to a screw that is coupled to the fixator.

9. A method as in claim 1, wherein the distraction member attaches to an outer wall of the fixator.

10. A method of distracting a disc space between a first vertebral body and a second vertebral body, comprising:
attaching a distraction member to a fixator that is implanted onto the first vertebral body while the fixator remains implanted on the first vertebral body, wherein the second vertebral body is not attached to the fixator;
distracting the disc space by exerting a distraction force onto the distraction member.

11. A method as in claim 10, further comprising:
evacuating the disc space; and
placing an orthopedic device in the evacuated disc space.

12. A method as in claim 10, further comprising:
evacuating the disc space; and
fusing the disc space.

13. A bone distraction device, comprising:
a member having a first portion and a second portion, the first portion adapted to couple to a bone fixator attached to a bone such that the first portion remains coupled to the bone fixator during distraction of a disc space, the second portion adapted to coupled onto a distractor platform that produces a distraction force sufficiently strong to distract a disc space, wherein the distraction device is adapted to transmit the distraction force produced by the distraction platform onto the implanted bone fixator and the bone to which the fixator is attached while the first portion remains coupled to the bone fixator.

14. A device as in claim 13, wherein the first portion is adapted to couple to a bone fastener of the bone fixator.

15. A device as in claim 13, wherein the first portion couples to a threaded aperture of the bone fixator.

16. A device as in claim 13, wherein the first portion couples to a non-threaded aperture of the bone fixator.

17. A device as in claim 13, wherein the first portion clamps onto a portion of the bone fixator.

18. A bone distraction device, comprising:
a member having a first portion and a second portion, the first portion adapted to couple to a bone fixator attached to a bone such that the first portion remains coupled to the bone fixator during distraction of a disc space, the second portion adapted to couple to a neighboring bone that is not attached to the fixator so that at least one disc space is situated between the bone attached to the fixator and the neighboring bone not attached to the fixator, wherein the distraction device is adapted to exert a force across the disc space while the first portion remains coupled to the bone fixator.

19. A device as in claim 18, wherein the first portion is adapted to couple to a bone fastener of the bone fixator.

20. A device as in claim 18, wherein the first portion couples to a threaded aperture of the bone fixator.

21. A device as in claim 18, wherein the first portion couples to a non-threaded aperture of the bone fixator.

22. A device as in claim 18, wherein the first portion clamps onto a portion of the bone fixator.

23. A method for the distraction of a disc space that is situated between a first vertebral body attached to a previously implanted bone fixator and a second vertebral bone that is not attached to the bone fixator, comprising:
attaching a distraction member onto an aperture of the previously implanted fixator while the fixator remains attached to the first vertebral body such that the fixator is not removed from the first vertebral body;
exerting a distraction force onto the fixator via the distraction member to distract the disc space.

24. A method as in claim 23, further comprising:
evacuating the disc space; and
placing an orthopedic device in the evacuated disc space.

25. A method as in claim 23, further comprising:
evacuating the disc space; and
fusing the disc space.

26. A method as in claim 23, wherein the distraction member is attached to a threaded aperture of the fixator.

27. A method as in claim 23, wherein the distraction member is attached to a non-threaded aperture of the fixator.

28. A method as in claim 23, wherein the distraction member comprises an elongate member and further comprising mating the elongate member with a distractor device that exerts the distraction force.

29. A method for the distraction of a disc space that is situated between a first vertebral body attached to a previously implanted bone fixator and a second vertebral bone that is not attached to the bone fixator, comprising:
clamping a distraction member onto the previously implanted fixator while the fixator remains attached to the first vertebral body such that the fixator is not removed from the first vertebral body;
exerting a distraction force onto the fixator via the distraction member to distract the disc space.

30. A method as in claim 29, further comprising:
evacuating the disc space; and
placing an orthopedic device in the evacuated disc space.

31. A method as in claim 29, further comprising:
evacuating the disc space; and
fusing the disc space.

32. A method as in claim 29, wherein the distraction member comprises an elongate member and further comprising mating the elongate member with a distractor device that exerts the distraction force.

33. A method as in claim 1, wherein the distraction member clamps onto an outer wall of the fixator.
